(19) United States
(12) Patent Application Publication
(10) Pub. No.: US 2023/0380906 A1
(43) Pub. Date: Nov. 30, 2023

(54) Ultrasound Imaging Device with Automatic Adjusting Needle Guide

(71) Applicant: Bard Access Systems, Inc., Salt Lake City, UT (US)

(72) Inventors: Anthony K. Misener, Bountiful, UT (US); Steffan Sowards, Salt Lake City, UT (US); William Robert McLaughlin, Bountiful, UT (US)

(21) Appl. No.: 17/825,976

(22) Filed: May 26, 2022

(51) Int. Cl. A61B 34/20; A61B 8/08; A61B 8/00

(52) U.S. Cl. CPC A61B 34/20; A61B 8/085; A61B 8/4245; A61B 2034/2063

(57) ABSTRACT

A medical device guide system that includes an ultrasound probe is configured for imaging portions of a patient's body. The system is configured to automatically position and/or orient a needle guide with respect to the probe in accordance with a desired needle trajectory with respect to the target blood vessel. The system includes one or more of a blood vessel detection system, a probe orientation system and/or a needle tracking system to facilitate automatically positioning of the needle guide. Some needle guides include multiple channels that define different angles of needle insertion. The needle guide is selectively coupled with a needle guide connector that is operatively coupled with the probe via an electro-mechanical mechanism.

### BACKGROUND

Treating a patient via the patient vasculature using a catheters or other vascular devices provides several benefits for the patient as well as the healthcare provider. In some instances, accessing the patient vasculature may be difficult and may also present some risks to the patient, such internal bleeding, for example. In some instances, blood vessels may also be difficult to identify. The systems and methods described herein address the forgoing.

### SUMMARY

Briefly summarized, disclosed herein is an ultrasound system that includes: an ultrasound probe having a head portion configured to obtain ultrasound image data; a guide connector coupled with the head portion; a needle guide coupled with the guide connector, where a position and/or an angle of the needle guide is adjustable with respect to the head portion. The needle guide includes a needle channel, and the needle channel is configured to define a lateral position and an angle of a needle disposed within the needle channel with respect to the needle guide.

In some embodiments, a longitudinal position of the needle guide with respect to the head portion is adjustable.

In some embodiments, an angle of the needle guide with respect to the head portion is adjustable.

In some embodiments, a longitudinal position of the guide connector with respect to the head portion is adjustable.

In some embodiments, an angle of the guide connector with respect to the head portion is adjustable.

In some embodiments, the system further includes: (i) an electro-mechanical guide adjustment mechanism coupled between the head portion and the needle guide; (ii) one or more processors; and (iii) a non-transitory computer-readable storage medium having logic stored thereon that, when executed by the one or more processors, performs operations that include automatically adjusting the position and/or the angle of the needle guide with respect to the head portion via the electro-mechanical guide adjustment mechanism.

In some embodiments, the operations further include (i) receiving a desired entry point and/or insertion angle of the needle from the clinician via a user input device of the ultrasound system and (ii) automatically adjusting the position and/or an angle of the needle guide with respect to the head portion based on the desired entry point and/or insertion angle.

In some embodiments, the needle guide includes two or more needle channels, where each needle channel defines an insertion angle that is different from the other needle channel(s), and where the operations further include (i) receiving a desired insertion angle from the user input device as entered by a clinician and (ii) automatically adjusting a lateral position of the needle guide to centrally align a needle channel of the two or more needle channels that defines the desired insertion angle.

In some embodiments, the ultrasound system further includes a probe orientation monitoring system configured to determine an orientation of the ultrasound probe, and the operations further include (i) receiving probe orientational data from the probe orientation monitoring system and (ii) automatically adjusting the angle of the needle guide with respect to the head portion based on the probe orientation data.

In some embodiments, the ultrasound system further includes a needle tracking system configured to determine a position and an angle of the needle when the needle is disposed within the needle channel, and the operations further include (i) receiving needle tracking data from the needle tracking system and (ii) automatically adjusting the position and/or the angle of the needle guide with respect to the head portion based on the needle tracking data.

In some embodiments, the ultrasound system further includes an anatomical target identification system configured to identify at least a target blood vessel, and the operations further include (i) receiving anatomical target identification data from the anatomical target identification system and (ii) automatically adjusting the position and/or an angle of the needle guide with respect to the head portion based on the anatomical target identification data. In some embodiments, the operations further include automatically adjusting the position and/or an angle of the needle guide to align the needle channel with the identified target blood vessel.

In some embodiments, the anatomical identification system configured to identify the target blood vessel and at least one other anatomical structure, and the operations further include automatically adjusting the position and/or an angle of the needle guide with respect to the head portion such that a longitudinal axis of the needle channel (i) intersects the target blood vessel and (ii) avoids the at least one other anatomical structure.

In some embodiments, the needle guide is positionably coupled with the guide connector via longitudinally oriented track such that positioning the needle guide along the track changes an entry point for the needle while maintaining a constant angle of the needle with respect to the head portion. In some embodiments, the operations include adjusting the position of the needle guide along the track.

Also disclosed herein is a method performed by an ultrasound probe for defining a vascular access pathway. According to some embodiments, the method includes (i) obtaining an ultrasound image data of a patient vasculature via the ultrasound probe, the ultrasound image data including an identification of at least a target blood vessel; and (ii) automatically adjusting a position and/or angle of a needle guide with respect to the ultrasound probe based on the ultrasound image data so that a longitudinal axis of the needle channel of the needle guide intersects the target blood vessel at a desired angle, the needle guide selectively coupled with the probe so as to be adjustable with respect to the probe via an electro-mechanical mechanism of the probe.

In some embodiments, the method further includes (i) receiving a desired entry point and/or insertion angle of the needle from the clinician via a user input device of the ultrasound probe and (ii) automatically adjusting the position and/or an angle of the needle guide with respect to the ultrasound probe based on the desired entry point and/or insertion angle.

In some embodiments of the method, the needle guide includes two or more needle channels, where each needle channel defines an angle with respect to the ultrasound probe that is different from the other needle channel(s), and the method further includes (i) receiving a desired angle from the clinician via the user input device and (ii) automatically adjusting a lateral position of the needle guide with respect to the probe to centrally align a needle channel of the two or more needle channels that defines the desired insertion angle.

In some embodiments of the method, the probe includes a probe orientation monitoring system configured to determine an orientation of the ultrasound probe, and the method further includes automatically adjusting the angle of the needle guide with respect to the ultrasound probe based on the orientation of the probe.

In some embodiments of the method, the ultrasound probe includes a needle tracking system configured to determine a position and an angle of the needle when the needle is disposed within the needle channel. In such an embodiment, the method further includes automatically adjusting the position and/or the angle of the needle guide with respect to the head portion based on the angle and/or position of the needle determined by the needle tracking system.

In some embodiments, the method further includes identifying the target blood vessel and at least one other anatomical structure, and automatically adjusting the position and/or an angle of the needle guide with respect to the ultrasound probe so that a longitudinal axis of the needle avoids the at least one other anatomical structure.

These and other features of embodiments of the present invention will become more fully apparent from the following description and appended claims, or may be learned by the practice of embodiments of the invention as set forth hereinafter.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 is simplified perspective view of an ultrasound imaging system, in accordance with some embodiments;

FIG. 2 is a front view of a handheld ultrasound probe of the system of FIG. 1, in accordance with some embodiments;

FIG. 3A is a side view of a head portion of the ultrasound probe of FIG. 1 illustrating a position-ability of a needle guide connector, in accordance with some embodiments;

FIG. 3B is the side view of the head portion of the ultrasound probe of FIG. 1 illustrating a rotatability of a needle guide connector, in accordance with some embodiments;

FIG. 4 is a perspective view of the needle guide for coupling with the needle guide connector of FIGS. 3A-3B, in according with some embodiments;

FIGS. 5A-5B illustrate the head portion of the ultrasound probe of FIG. 1 in contact with a skin surface of a patient, where the needle guide of FIG. 4, having a needle coupled thereto, is coupled with the needle guide connector of FIGS. 3A-3B, and where the position-ability and rotatability, respectively, are shown with respect to a blood vessel, in accordance with some embodiments;

FIG. 6A illustrates a needle guide that is manually positionable with respect to the needle guide connector, in accordance with some embodiments;

FIG. 6B illustrates a needle guide that is manually rotatable with respect to the needle guide connector, in accordance with some embodiments;

FIG. 7A illustrates a needle guide having multiple needle channels, in accordance with some embodiments;

FIG. 7B illustrates the needle guide of FIG. 7A coupled with the needle guide connector, where lateral displacement of the needle guide connector laterally displaces the needle guide, in accordance with some embodiments;

FIG. 7C illustrates another embodiment of the needle guide of FIG. 7A slidably coupled with the needle guide connector, in accordance with some embodiments; and

FIG. 8 is a block diagram of logic operations of the system of FIG. 1, in accordance with some embodiments.

### DETAILED DESCRIPTION

Reference will now be made to figures wherein like structures will be provided with like reference designations. It is understood that the drawings are diagrammatic and schematic representations of exemplary embodiments of the present invention, and are neither limiting nor necessarily drawn to scale.

For clarity it is to be understood that the word “proximal” refers to a direction relatively closer to a clinician using the device to be described herein, while the word “distal” refers to a direction relatively further from the clinician. For example, the end of a needle or catheter placed within the body of a patient is considered a distal end of the needle or catheter, while the needle or catheter end remaining outside the body is a proximal end of the needle or catheter. Also, the words “including,” “has,” and “having,” as used herein, including the claims, shall have the same meaning as the word “comprising.”

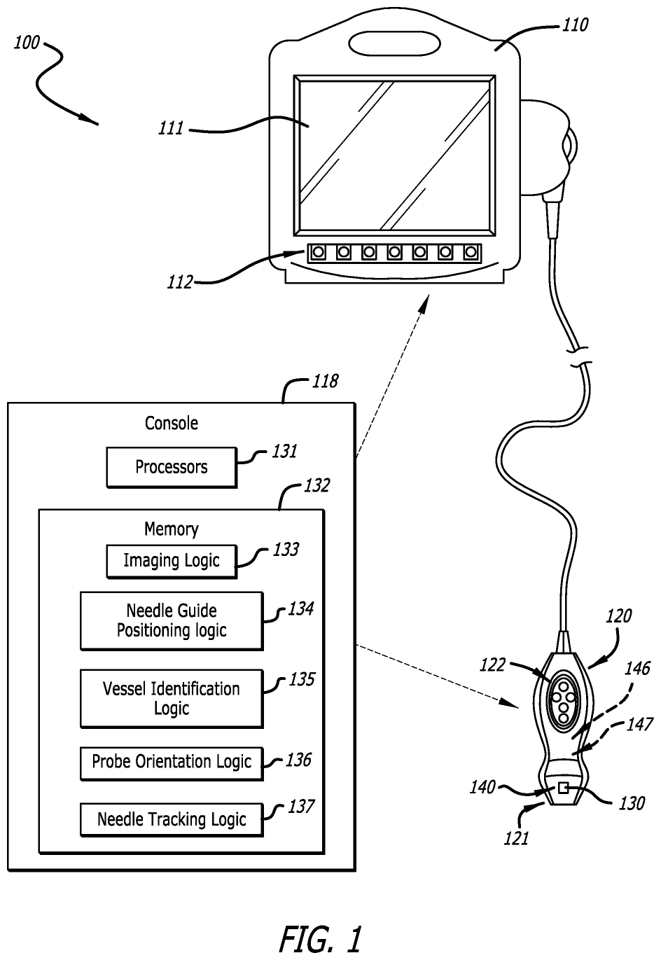

FIG. 1 illustrates an ultrasound imaging system (system) 100 for ultrasonically imaging portions of a patient body. The system 100 is generally configured to facilitate accessing a patient vasculature such as defining a vascular access pathway for a needle. The system may further facilitate the positioning and/or orienting of a needle so that the needle may be inserted along the vascular access pathway into the vasculature.

The system 100 generally includes a display module 110, an ultrasound probe (probe) 120, and a console 130. The display module 110 includes a display 111. The display 111 and the probe 120 each include one or more user input controls 112, 122, respectively. The console includes one or more processors 131 and memory 132 (e.g., non-volatile memory or non-transitory, computer-readable medium) having logic stored thereon. Any portion of the console 130 may be included in the display module 110 and/or the probe 120. The logic includes imaging logic 133, needle guide positioning logic 134, and vessel detection logic. The probe 120 may optionally include a probe orientation monitoring system 146 and/or a needle tracking system 147, where the logic may also optionally include probe orientation logic 136 and/or needle tracking logic 137. Briefly, the probe 120 is configured to (i) transmit ultrasonic signals from a head portion 121 thereof into a portion of a patient body and (ii) receive the ultrasonic signals after reflection by internal structures of the patient body. The system 100 processes the reflected ultrasonic signals for depiction on the display 111. Needle guidance systems are taught by U.S. Pat. No. 10,231,697 titled “NEEDLE GUIDES FOR A SONOGRAPHIC IMAGING DEVICE” filed on May 2, 2013, which is incorporated herein by reference in its entirety.

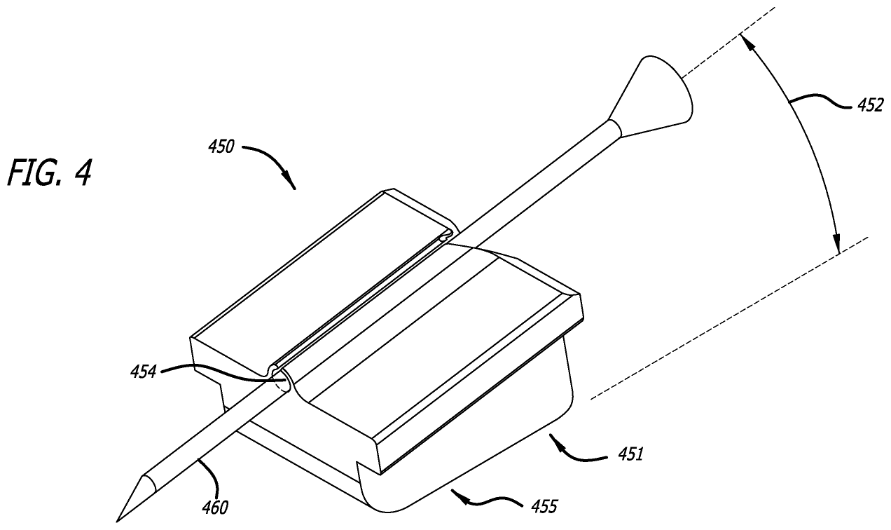

The probe 120 includes a needle guide connector 130 that during use is coupled with a needle guide (see needle guide 450 of FIG. 4). The probe 120 includes an electro-mechanical mechanism 140 that is operatively coupled with the needle guide connector 130 as further described below.

The optional probe orientation monitoring system 146 is generally configured to monitor the position and/or movement of the probe 120 during use. The probe orientation monitoring system 146 may include a gyroscope and/or an inertia measurement unit (IMU) to determine the orientation and/or an orientation disturbance of the probe 120, and the probe orientation monitoring system 146 provides probe orientation data to the probe orientation logic 136. In some embodiments, the probe orientation monitoring system 146 may determine a position and/or displacement of the probe 120 during use.

The optional needle tracking system 147 is generally configured to monitor (e.g., magnetically monitor) the position and/or orientation of a needle with respect to the probe 120 during use. In some embodiments, the needle may include a number of magnetic elements and the ultrasound probe 120 may include a number of magnetic sensors. Medical device tracking and guidance is described for various instruments, including needles, in U.S. Pat. No. 9,521,961 titled “systems and methods for guiding a medical instrument” filed on Dec. 23, 2011, which is included herein by reference in its entirety. The needle tracking system 147 provides needle tracking data to the needle tracking logic 137. In some embodiments, the system 100 may include the needle having magnetic elements.

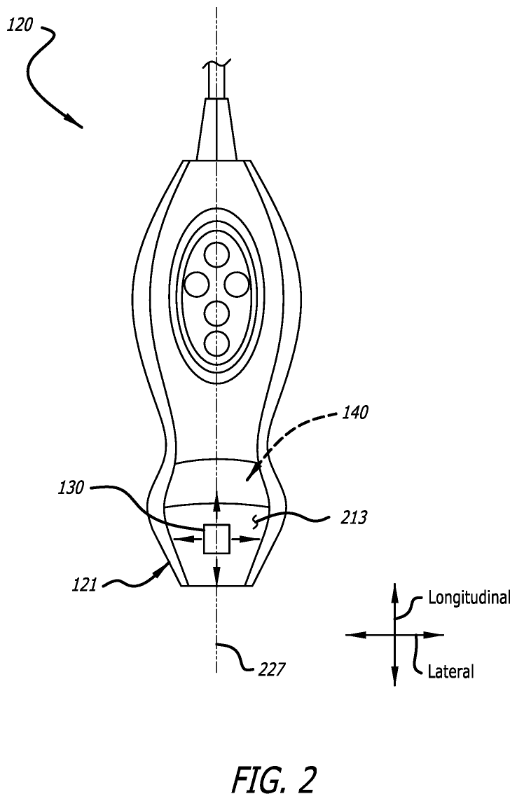

FIG. 2 illustrates a front view of the probe 120. The probe 120 defines a longitudinal axis 227 and the longitudinal axis 227 may also define an imaging axis of the head portion 121. In the illustrated embodiment, the needle guide connector 130 is located on the head portion 121. In the illustrated embodiment, the needle guide connector 130 is located on a front side 223 of the probe 120. In other embodiments, the needle guide connector 130 may be located on side other than the front side 223. The needle guide connector 130 includes coupling features to facilitate the selective coupling and decoupling of the needle guide connector 130 with the needle guide, such as hooks, protrusions, depressions, guideways, and the like. In some embodiments, the needle guide connector 130 may rigidly couple with the needle guide so that a position of the needle guide connector 130 with respect to the head portion 121 defines the position of the needle guide with respect to the longitudinal axis 227. Similarly, in some embodiments, the needle guide connector 130 may rigidly couple with the needle guide so that an orientation of the needle guide connector 130 with respect to the head portion 121 defines the orientation of the needle guide with respect to the longitudinal axis 227.

The probe 120 includes the electro-mechanical mechanism 140 of which all or a portion thereof may be disposed within a housing of the probe 120. The electro-mechanical mechanism 140 is operative coupled with the needle guide connector 130 such that operation of the electro-mechanical mechanism 140 adjusts a position and/or orientation of the needle guide connector 130 with respect to the head portion 121/longitudinal axis 227. The electro-mechanical mechanism 140 may include a number of electro-mechanical actuators, such as rotational motors, linear motors, or solenoids, for example along with other mechanical elements to facilitate adjustment of the position and/or orientation of the needle guide connector 130 based on electrical signals defined by the guide positioning logic 134. The electro-mechanical mechanism 140 may be configured to adjust one or more of a longitudinal position, a lateral position, or orientation of the needle guide connector 130 with respect to the head portion 121/longitudinal axis 227, where the orientation includes an angle of the needle guide connector 130 with respect to the longitudinal axis 227.

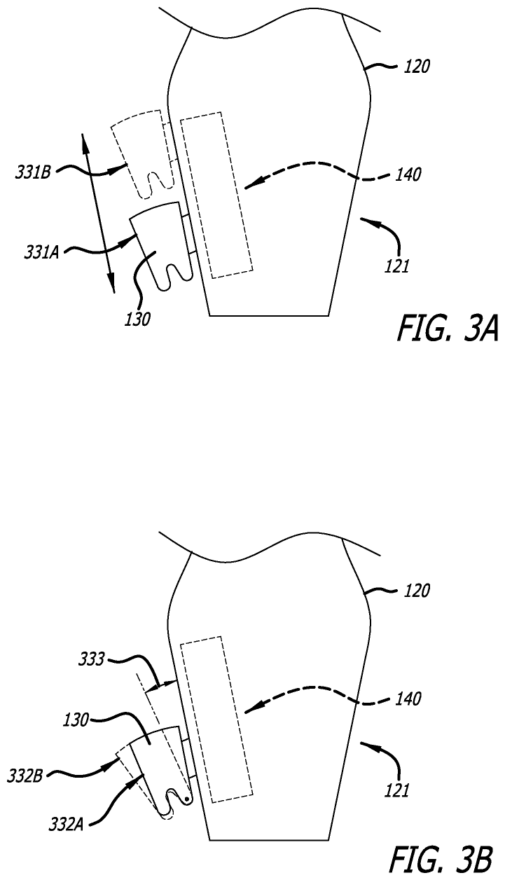

FIG. 3A illustrates a side view of the head portion 121 of the probe 120 depicting the needle guide connector 130 as positionable along the probe 120. As stated above the electro-mechanical mechanism 140 may be configured to displace the needle guide connector 130 longitudinally with respect to the head portion 121. FIG. 3A shows the needle guide connector 130 in a first longitudinal position 331A and second longitudinal position 331B (in phantom lines). In some embodiments, the electro-mechanical mechanism 140 may be configured to position the needle guide connector 130 at a number (e.g., 2, 3, 4 or more) of discreet positions. In other embodiments, the electro-mechanical mechanism 140 may be configured to continuously position needle guide connector 130 along a travel length, such as a length between the first longitudinal position 331A and second longitudinal position 331B, for example.

FIG. 3B illustrates a side view of the head portion 121 of the probe 120 depicting the needle guide connector 130 as rotatable with respect to the probe 120. As stated above, the electro-mechanical mechanism 140 may be configured to orient the needle guide connector 130 (i.e., rotate the needle guide connector across an angular range 333) with respect to the head portion 121. FIG. 3B shows the needle guide connector 130 in a first orientation 332A and second orientation 332B (in phantom lines). In some embodiments, the electro-mechanical mechanism 140 may be configured to rotate the needle guide connector 130 to a number (e.g., 2, 3, 4 or more) of discreet orientations. In other embodiments, the electro-mechanical mechanism 140 may be configured to continuously rotate needle guide connector 130 across the angular range 333, such as between the first orientation 332A and second orientation 332B, for example.

FIG. 4 is a perspective illustration of an exemplary needle guide 450. The needle guide 450 is configured to selectively couple with the needle guide connector 130. More specifically, the needle guide 450 may be coupled with and decoupled from the needle guide connector 130 by the clinician during use. In some embodiments, the needle guide 450 includes a pocket (or recessed portion) 455 disposed on an underside of the needle guide 450. The pocket 455 is configured to receive the needle guide connector 130 or a portion thereof to facilitate the coupling of the needle guide 450 with the needle guide connector 130.

The needle guide 450 includes a needle channel 454. The needle channel 454 is configured to slidably and longitudinally receive a needle 460 therein. During use, the clinician may insert the needle 460 into the needle channel 454. The needle channel 454 is sized to constrain an orientation and lateral position of the needle 460 with respect to the needle guide 450 while permitting longitudinally sliding displacement of the needle 460 therealong. The needle guide 450 may be one of a plurality of needle guides 450 that may be employed with the probe 120, where the channel 454 of each needle guide 450 may be sized to receive (i.e., fit) a needle 460 of a different size (e.g., diameter). The needle channel 454 defines an angle 452 with respect to a coupling datum 451. The coupling datum 451 is configured to define a fixed angle of the needle channel 454 with respect to the needle guide connector 130 of the probe 120 when the needle guide 450 is coupled with the needle guide connector 130. In some embodiments, the plurality of needle guides 450 includes needle guides 450 that define different angles 452.

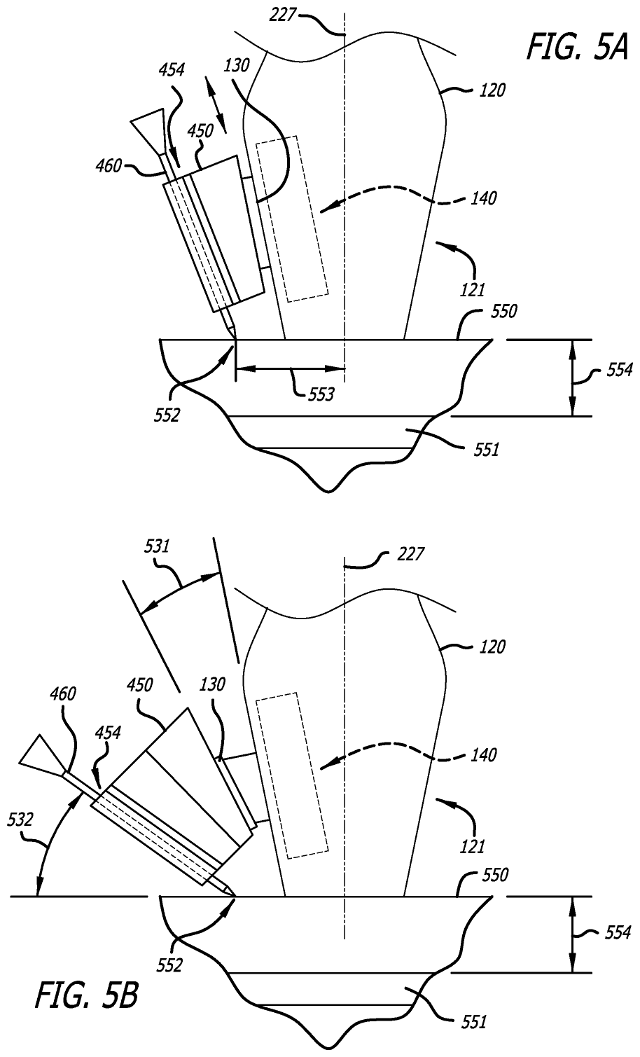

FIGS. 5A-5B illustrate a side view of the head portion 121 of the probe 120 in contact with a skin surface 550 of a patient, where a blood vessel 551 is disposed a subcutaneous depth 554 from the skin surface 550. The needle guide 450 is rigidly coupled with the needle guide connector 130, i.e., so that the needle guide 450 defines a fixed position and orientation with the respect to the needle guide connector 130. The needle 460 is disposed within the needle channel 454 of the needle guide 450. The electro-mechanical mechanism 140 is coupled with the needle guide connector 130.

Referring to FIG. 5A, the electro-mechanical mechanism 140 is configured to longitudinally displace the needle guide 450 along the probe 120 (i.e., up and down with respect to the head portion 121 or, more broadly, away from and toward the skin surface 550 during use). The longitudinal axis 227 of the probe 120 is shown for reference, where the longitudinal axis 227 may also represent an imaging axis of the probe 120. As illustrated, the needle 460 is positioned to engage the skin surface 550 at an entry point 552 located at a distance 553 away from the longitudinal axis 227. As may be appreciated by one of ordinary skill, longitudinally displacing the needle guide 450 along the probe 120 changes the distance 553. More specifically, raising the needle guide 450 moves the entry point closer to the longitudinal axis 227, and lowering the needle guide 450 moves the entry point further from the longitudinal axis 227.

The logic 123 (FIG. 1) may be configured to define the longitudinal position of the needle guide 450 based on a distance defined 553. By way of one example, in use, the clinician may enter an entry point location (e.g., a desired distance 553) into the system 100 and the logic 123 may cause the electro-mechanical mechanism 140 to longitudinally displace the needle guide connector 130 to a location consistent with the desired entry point.

With reference to FIG. 5B, the electro-mechanical mechanism 140 is configured to rotate the needle guide 450 with respect to the longitudinal axis 227 of the probe 120, e.g., adjust the angle 531. As illustrated, the needle 460 is positioned to engage the skin surface 550 at an insertion angle 532. As may be appreciated by one of ordinary skill, adjusting the angle 531 also adjusts the insertion angle 532. More specifically, increasing the angle 531 reduces the insertion angle 532 and decreasing the angle 531 increases the insertion angle 532.

The needle guide positioning logic 134 (FIG. 1) may be configured to define the angle 531 based on a desired insertion angle 532. By way of one example, in use, the clinician may enter a desired insertion angle 532 into the system 100 and the needle guide positioning logic 134 may cause the electro-mechanical mechanism 140 to rotate the needle guide connector 130 to an angle 531 consistent with the desired insertion angle 532.

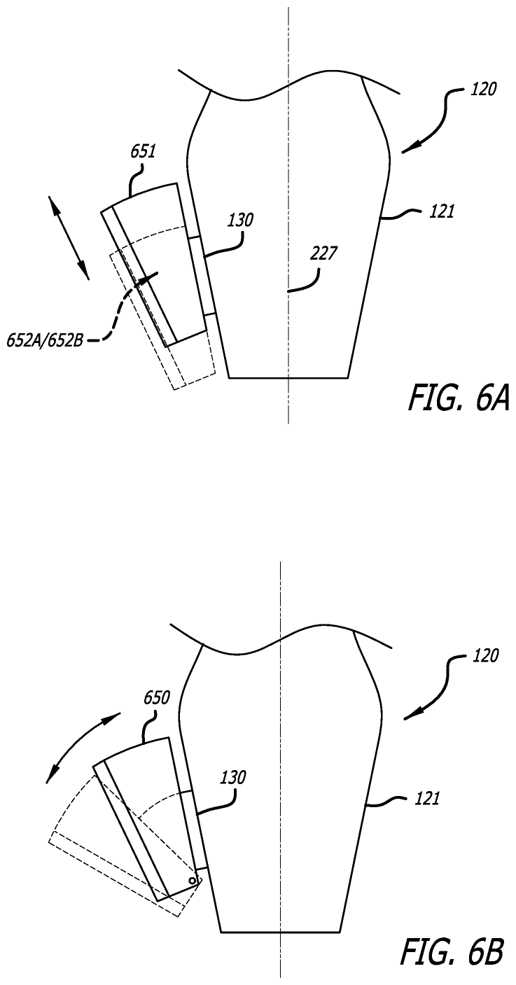

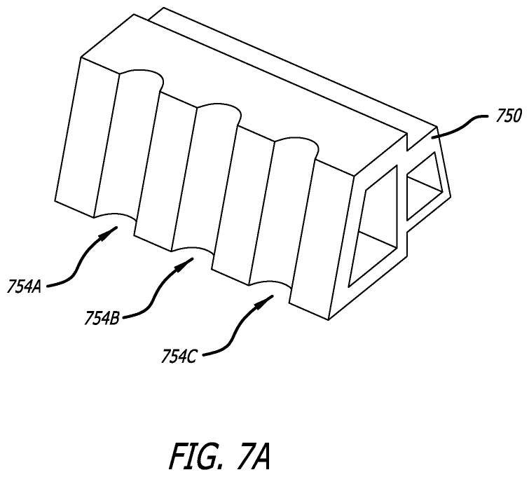

FIG. 6A illustrates a side view of the head portion 121 of the probe 120 having a needle guide 650 which in some respects may resemble the needle 450 of FIG. 4. The needle guide 650 is slidably coupled with the needle guide connector 130, i.e., so that the needle guide 650 defines a fixed lateral position and a fixed orientation with the respect to the needle guide connector 130 while providing for longitudinal slidable displacement of the needle guide 650 with respect to the needle guide connector 130. By way of example, the needle guide 650 may include a first track member 652A configured to slidably engage with a corresponding second track member 652B of the needle guide connector 130 thereby facilitating longitudinal slidable engagement. As discussed above in relation to FIG. 5A, raising the needle guide 650 moves the entry point 552 (FIG. 5A) of the needle closer to the longitudinal axis 227 of the probe 120, and lowering the needle guide 650 moves the entry point further from the longitudinal axis 227 of the probe 120.

In use, the clinician may manually position the needle guide 650 with respect to the needle guide connector to define the entry point 552 of the needle 460. In some embodiments, the needle guide positioning logic 134 (FIG. 1) may also adjust the longitudinal position of the needle guide connector 130 to further or alternatively define the entry point 552. By way of one example, in use, the clinician may manually position the needle guide 650 with respect to the needle guide connector 130 to define an initial or rough entry point and the electro-mechanical mechanism 140 may longitudinally displace the needle guide connector 130 to further define the entry point.

FIG. 6B illustrates a side view of the head portion 121 of the probe 120 having a needle guide 651 which in some respects may resemble the needle 450 of FIG. 4. The needle guide 651 is rotatably coupled with the needle guide connector 130, i.e., so that the needle guide 651 defines a fixed lateral position and a fixed longitudinal position the respect to the needle guide connector 130 while providing for an adjustable orientation of the needle guide 651 with respect to the needle guide connector 130. As discussed above in relation to FIG. 5B, rotating the needle guide 651 adjusts the insertion angle 532 (FIG. 5B) of the needle 460 (FIG. 5B) with respect the skin surface 550 of the patient.

In use, the clinician may manually adjust the orientation of the needle guide 651 with respect to the needle guide connector 130 to define the insertion angle 532 of the needle 460 with respect to the skin surface 550. In some embodiments, in use, the clinician may manually rotate the needle guide 650 with respect to the needle guide connector 130 to define an initial or rough insertion angle 532 and the electro-mechanical mechanism 140 may rotate the needle guide connector 130 to further define the insertion angle.

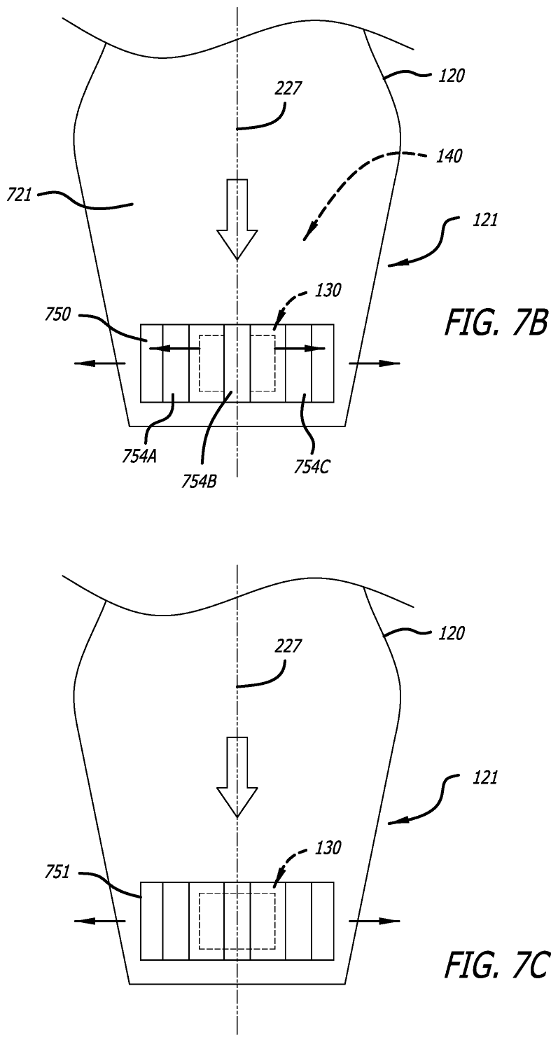

FIG. 7A illustrates a needle guide 750 having a number (e.g., 2, 3, or more) of needle channels. By way of example, the needle guide 750 includes three needle channels 754A-754B. Each of the needle channels 754A-754B define a different insertion angle for a needle inserted therethrough. As such, the clinician may define the insertion angle 532 (FIG. 5B) by choosing the respective needle channel. The needle guide 750 is configured for lateral positioning with respect to the probe 120. The needle guide 750 is configured for coupling with the needle guide connector 130 as further described below. A needle guide with multiple needle channels is taught by U.S. Pat. No. 9,788,812 titled “NEEDLE GUIDE WITH SELECTABLE ASPECTS” filed on Jun. 22, 2012, which is incorporated herein by reference in its entirety.

FIG. 7B illustrates a front view of the head portion 121 of the probe 120. The needle guide 750 is rigidly coupled with the needle guide connector 130 so that a longitudinal position, a lateral position, and an orientation of the needle guide 750 with respect to the needle guide connector 130 are fixed. In the illustrated embodiment, the electro-mechanical mechanism 140 is configured to adjust a lateral position of the needle guide connector 130 across the front face 721 of the probe 120 such any one of the needle channels 754A-754B may be positioned in alignment with the longitudinal axis 227 of the probe 120.

The needle guide positioning logic 134 may be configured to cause the electro-mechanical mechanism 140 to laterally displace the needle guide connector 130 so as to align a desired needle channel with the longitudinal axis 227. By way of example, the clinician may input a desired insertion angle 532 consistent with the needle channel 754C into the system 100, and in response, the needle guide positioning logic 134 may cause the electro-mechanical mechanism 140 to laterally displace the needle guide connector 130 such that the needle channel 754C is aligned with the longitudinal axis 227.

FIG. 7C illustrates a front view of the head portion 121 of the probe 120 including a needle guide 751 that may in some respects resemble the needle guide 750. The needle guide 751 is coupled with the needle guide connector 130 so that a longitudinal position and an orientation of the needle guide 750 with respect to the needle guide connector 130 are fixed, and so that the lateral position of the needle guide 751 with respect to the needle guide connector 130 is adjustable, such as along a laterally oriented track. In use, the clinician may manually displace the needle guide 751 laterally with respect to the needle guide connector 130 so that the needle channel consistent a desired insertion angle 532 is aligned with the longitudinal axis 227. By way of example, the clinician may define a desired insertion angle 532 consistent with the needle channel 754C. The clinician may then manually displace the needle guide connector 130 such that the needle channel 754C is aligned with the longitudinal axis 227.

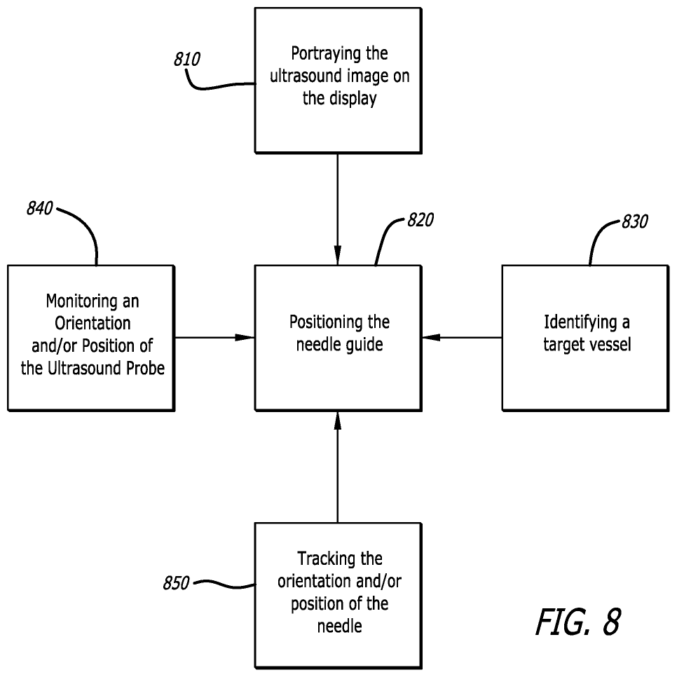

FIG. 8 is a block diagram illustrating logic operations of the system 100. The imaging logic 133 may obtain ultrasound imaging data from the head portion 121 (block 810). The imaging logic 133 may portray an ultrasound image of a target area of the patient on the display 111. The ultrasound image may include anatomical structures of the target area. For example, the imaging logic 133 may obtain ultrasound image of a target area of an arm of the patient. The image of the target area may include anatomical structures, such as blood vessels, bones, or nerve bundles, for example. During use, the clinician may review the ultrasound image on the display 111. In response to the review, the clinician may visually identify a target blood vessel for the insertion of the needle 460 therein. The clinician may also visually identify anatomical structures to avoid when accessing the target blood vessel. In further response, the clinician may define the entry point 552 for the needle 460 and/or the insertion angle 532 of the needle 460. In some embodiments, the clinician may enter a number of vascular access parameters into the system, such as the entry point 552 and/or the insertion angle 532.

The needle guide positioning logic 134 may adjust the position and/or orientation of the needle guide connector 130 (block 820). More specifically, the needle guide positioning logic 134 may cause the electro-mechanical mechanism 140 to move the needle guide connector 130. The needle guide positioning logic 134 may adjust the position and/or orientation of the needle guide connector 130 based on vascular access parameters stored in memory 132. The vascular access parameters may include standard values, such as a common insertion angle, for example. The vascular access parameters may also be input by the clinician or obtained automatically as further described below.

The vessel identification logic 135 may automatically identify one or more anatomical structures within the ultrasound image (block 830). For example, the vessel identification logic 135 may identify a target blood vessel. Identifying a target blood vessel may include differentiating a blood vessel from a bone, a nerve bundle, or other anatomical structure. Identifying a target blood vessel may further include differentiating a vein from an artery. The vessel identification logic 135 may also determine a subcutaneous depth 554 (FIGS. 5A-5B) of the target blood vessel. The vessel identification logic 135 may further determine a position of the target blood vessel in relation to other anatomical structures. As a result of the identifications and determinations, the vessel identification logic 135 may define the entry point 552 and/or the insertion angle 532 for the guide positioning logic 134.

In some embodiments, the vessel identification logic 135 may identify anatomical structures to be avoided during insertion of the needle 460. In such embodiments, the vessel identification logic 135 may define the entry point 552 and/or the insertion angle 532 for the needle 460 that avoids the anatomical structures to be avoided.

The probe orientation logic 136 may monitor the position and/orientation of the ultrasound probe 120 during use (block 840). In some instances, the clinician may disturb the position and/or orientation of the ultrasound probe 120 during the needle insertion process leading to an error in the entry point 552 and/or insertion angle 532 of the needle 460. The probe orientation logic 136 may determine a shift in the position/orientation of the probe 120 and provide probe shift data to the needle guide positioning logic 134. The needle guide positioning logic 134 may then adjust the position/orientation of the needle guide connector 130 based on the shaft data to maintain the defined entry point 552 and/or insertion angle 532.

The needle tracking logic 137 may monitor/track the position/orientation of the needle 460 with respect to the probe 120 during use (block 850). More specifically, the needle tracking logic 137 may receive needle tracking data from the needle tracking system 147. The needle tracking logic 137 may then determine position/orientation of the needle 460 in relation to the defined entry point 552 and the insertion angle 532. In response, the needle tracking logic 137 may provide a notice to the clinician that the needle 460 is or is not correctly positioned and/or oriented in relation to the defined entry point 552 and the insertion angle 532. In some embodiments, the needle tracking logic 137 may provide needle tracking data to the needle guide positioning guide logic 134 and in response, the needle guide positioning guide logic 134 may adjust the position and/or orientation of the needle guide connector 130 so that the needle 460 is correctly positioned in relation to the defined entry point 552 and the insertion angle 532. In some embodiments, the needle tracking logic 137 may also track a subcutaneous depth of the needle tip in relation to the subcutaneous depth 554 of the target blood vessel 551, and in response, the needle tracking logic 137 may provide a notice to the clinician that the needle tip is or is not located at a depth consistent with the subcutaneous depth 554 of the target blood vessel 551.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

1. An ultrasound system, comprising:
an ultrasound probe having a head portion configured to obtain ultrasound image data;
a guide connector coupled with the head portion; and
a needle guide coupled with the guide connector, the needle guide including a needle channel, the needle channel configured to define a lateral position and an angle of a needle disposed within the needle channel with respect to the needle guide,
wherein a position and/or an angle of the needle guide is adjustable with respect to the head portion.

2. The ultrasound system of claim 1, wherein a longitudinal position of the needle guide with respect to the head portion is adjustable.

3. The ultrasound system of claim 1, wherein an angle of the needle guide with respect to the head portion is adjustable.

4. The ultrasound system of claim 1, wherein a longitudinal position of the guide connector with respect to the head portion is adjustable.

5. The ultrasound system of claim 1, wherein an angle of the guide connector with respect to the head portion is adjustable.

6. The ultrasound system of claim 1, further comprising:
an electro-mechanical guide adjustment mechanism coupled between the head portion and the needle guide;
one or more processors; and
a non-transitory computer-readable storage medium having logic stored thereon that, when executed by the one or more processors, performs operations that include automatically adjusting the position and/or the angle of the needle guide connector with respect to the head portion via the electro-mechanical guide adjustment mechanism.

7. The ultrasound system of claim 6, wherein the operations further include:
receiving a desired entry point and/or insertion angle of the needle from the clinician via a user input device of the system, and
automatically adjusting the position and/or an angle of the needle guide with respect to the head portion based on the desired entry point and/or the insertion angle.

8. The ultrasound system of claim 7, wherein the needle guide includes two or more needle channels, each needle channel defining an insertion angle that is different from the other needle channel(s), wherein the operations further include:
receiving a desired insertion angle from the user input device as entered by a clinician, and
automatically adjusting a lateral position of the needle guide to centrally align a needle channel of the two or more needle channels that defines the desired insertion angle.

9. The ultrasound system of claim 6, further comprising a probe orientation monitoring system configured to determine at least an orientation of the ultrasound probe, wherein the operations further include:
receiving probe orientation data from the probe orientation monitoring system, and automatically adjusting the angle of the needle guide with respect to the head portion based on the probe orientation data.

10. The ultrasound system of claim 6, further comprising a needle tracking system configured to determine a position and an angle of the needle with respect to the ultrasound probe when the needle is disposed within the needle channel, wherein the operations further include:
receiving needle tracking data from the needle tracking system, and
automatically adjusting the position and/or the angle of the needle guide with respect to the head portion based on the needle tracking data.

11. The ultrasound system of claim 6, further comprising an anatomical target identification system configured to identify at least a target blood vessel, wherein the operations further include:
receiving anatomical target identification data from the anatomical target identification system, and
automatically adjusting the position and/or an angle of the needle guide with respect to the head portion based on the anatomical target identification data.

12. The ultrasound system of claim 11, wherein operations further include automatically adjusting the position and/or an angle of the needle guide with respect to the head portion to define an entry point and/or an insertion angle of the needle with respect to the identified target blood vessel.

13. The ultrasound system of claim 12, wherein
the anatomical identification system is configured to identify the target blood vessel and at least one other anatomical structure, and
the operations further include automatically adjusting the position and/or an angle of the needle guide with respect to the head portion to further define the entry point and/or the insertion angle of the needle such a longitudinal axis of the needle avoids the at least one other anatomical structure.

14. The ultrasound system of claim 1, wherein the needle guide is positionably coupled with the guide connector via a longitudinally oriented track such that positioning the needle guide along the track changes an entry point for the needle while maintaining a constant angle of the needle with respect to the head portion.

15. The ultrasound system of claim 14, wherein during use the position of the needle guide is manually adjusted along the track by the clinician.

16. A method performed by an ultrasound system for defining a vascular access pathway, comprising:
obtaining ultrasound image data of a patient vasculature via an ultrasound probe of the system, the ultrasound image data including an identification of at least a target blood vessel; and
automatically adjusting a position and/or angle of a needle guide with respect to the ultrasound probe based on the ultrasound image data so that a longitudinal axis of a needle within a needle channel of the needle guide intersects the target blood vessel at a desired insertion angle, the needle guide selectively coupled with the ultrasound probe so as to be adjustable with respect to the ultrasound probe via an electro-mechanical mechanism of the ultrasound probe.

17. The method of claim 16, further comprising:
receiving a desired entry point and/or insertion angle of the needle from the clinician via a user input device of the ultrasound system, and
automatically adjusting the position and/or an angle of the needle guide with respect to the ultrasound probe based on the desired entry point and/or insertion angle.

18. The method of claim 16, wherein the needle guide includes two or more needle channels, each needle channel defining an insertion angle that is different from the other needle channel(s), the method further comprising:
receiving a desired insertion angle from the clinician via the user input device; and
automatically adjusting a lateral position of the needle guide with respect to the ultrasound probe to centrally align a needle channel of the two or more needle channels that defines the desired insertion angle.

19. The method of claim 16, wherein the ultrasound probe includes a probe orientation monitoring system configured to determine an orientation of the ultrasound probe, the method further comprising automatically adjusting the angle of the needle guide with respect to the ultrasound probe based on the orientation of the ultrasound probe determined by the probe orientation monitoring system.

20. The method of claim 16, wherein the ultrasound probe includes a needle tracking system configured to determine a position and an angle of the needle when the needle is disposed within the needle channel, the method further comprising automatically adjusting the position and/or the angle of the needle guide with respect to the ultrasound probe based on the angle and/or position of the needle determined by the needle tracking system.

21. The method of claim 16, further comprising:
identifying the target blood vessel and at least one other anatomical structure within the ultrasound image data, and
automatically adjusting the position and/or an angle of the needle guide with respect to the ultrasound probe so that a longitudinal axis of the needle avoids the at least one other anatomical structure.
